(19) United States
(12) Patent Application Publication
(10) Pub. No.: US 2017/0020406 A1
(43) Pub. Date: Jan. 26, 2017

(54) METHOD FOR MEASURING BIOLOGICAL STIMULUS SIGNAL

(71) Applicant: UE Technology, Taichung (TW)

(72) Inventors: MIN-HUI CHIOUCHANG, Taichung (TW); JENG-REN DUANN, Taichung (TW); SHENG-CHUAN LIANG, Taichung (TW); YUNG-JIUN LIN, Taichung (TW); SHIH-CHE LO, Taichung (TW)

(21) Appl. No.: 15/287,252

(22) Filed: Oct. 6, 2016

Related U.S. Application Data
(63) Continuation-in-part of application No. 13/446,188, filed on Apr. 13, 2012.

(51) Int. Cl. A61B 5/0484; A61B 5/04; A61B 5/0478; A61B 5/00

(52) U.S. Cl. CPC A61B 5/0484; A61B 5/0006; A61B 5/04017; A61B 5/0478; A61B 5/7203; H04W 4/008

(57) ABSTRACT

The present invention discloses a method for measuring a biological stimulus signal, which replaces the conventional wired synchronized signal transmission technology with a wireless synchronized signal transmission technology to increase convenience in usage, and which compensates the delay time of wireless transmission in a calibration way to achieve a synchronization effect, whereby the stimulus signal can be corresponding to the response signal correctly, and whereby the synchronized integrated data of the subject can be correctly generated.

This application is a continuation-in-part, and claims priority, of from U.S. patent application Ser. No. 13/446,188 filed on Apr. 13, 2012, entitled “MEASURING METHOD FOR SYNCHRONIZING BIO-SIGNALS WITH STIMULATIONS”, the entire contents of which are hereby incorporated by reference.

### FIELD OF THE INVENTION

The present invention relates to a method for measuring a biological stimulus signal, particularly to a synchronization procession method for measuring a biological stimulus signal.

### BACKGROUND OF THE INVENTION

In the conventional method for measuring a biological stimulus signal, a trigger signal is input into a stimulation device to generate a stimulus signal. The stimulus signal may be a sound or a light beam, stimulating a subject in a special way. Suppose the subject is a human body. If the stimulus signal is a sound, the stimulus signal vibrates the ear drum. If the stimulus signal is a light beam, the stimulus signal stimulates the retina. Receiving a stimulus signal, the human body will generate a physiological response signal. The response signal will be detected by a sensor and directly stored into a signal processing device. The signal processing device is connected with the stimulation device by a transmission line, whereby the signal processing device can acquire the synchronization signal of the stimulation device.

Via comparing and integrating the response signal and the synchronization signal of the stimulus signal, the related data of the subject is acquired and used to evaluate the response of the subject. However, the conventional technology that uses the transmission lines to connect the stimulation device and the signal processing device limits the configuration of the stimulation device and the signal processing device and constrains the freedom of movement of the subject. While there are a plurality of biological subjects and a plurality of sensors, the wiring will be very complicated, inconvenient for the users and unfavorable to application.

### SUMMARY OF THE INVENTION

The primary objective of the present invention is to disclose a method for measuring a biological stimulus signal, which uses wireless transmission to increase convenience of usage and uses synchronized procession to make the stimulus signal correctly corresponding to the response signal, whereby to generate synchronized integrated data of the subject.

To achieve the abovementioned objective, the present invention comprises following steps.

Firstly, in order to acquire a time lag between wireless transmission and wired transmission, let a stimulation device provide a calibration signal and use a wireless transmission path and a wired transmission path to transmit the calibration signal to a signal reception-storage device, wherein the signal reception-storage device receives the calibration signals respectively coining from the wireless transmission path and the wired transmission path and separately delayed by a wireless transmission time and a wired transmission time, and wherein the difference between the wireless transmission time and the wired transmission time is the time lag. After the time lag is acquired, the wired transmission interface is removed.

Next, let the stimulation device provide a stimulus signal to a subject and simultaneously transmit a synchronization signal to the signal reception-storage device through a first wireless transmission interface, wherein the signal reception-storage device stores the synchronization signal as a comparison signal.

Next, after the subject receives the stimulus signal and generates a response, let a sensation device detect and converts the response and transmit the response to the signal reception-storage device to store as a response signal

Next, let a signal processing device, which is connected with the signal reception-storage device, use the time lag to calibrate either of the comparison signal and the response signal. After calibration, the signal processing device integrates the comparison signal and the response signal to generate a synchronized integrated data of the subject.

Therefore, the present invention is characterized in
1. The present invention adopts a wireless transmission technology, exempting the subject from being constrained in the space and distance of movements, applying to more types of tests, and increasing the convenience of usage.
2. The present invention uses the time lag to undertake calibration, avoiding the problem caused by the difference between the time of transmitting a signal wirelessly and the time of passing the signal through the sensation device, and acquiring the synchronized integrated data.

### BRIEF DESCRIPTION OF THE DRAWINGS

FIG. 1 is a block diagram schematically showing a system realizing a method for measuring a biological stimulus signal according to one embodiment of the present invention;

FIG. 2 is a diagram schematically showing a calibration signal according to one embodiment of the present invention;

FIG. 3 is a diagram schematically showing a stimulus signal and a pre-calibration response signal according to one embodiment of the present invention;

FIG. 4 is a diagram schematically showing a stimulus signal and a response signal after calibration according to one embodiment of the present invention; and

FIG. 5 is a diagram schematically showing a stimulus signal and a response signal after calibration according to another embodiment of the present invention.

### DETAILED DESCRIPTION OF THE PREFERRED EMBODIMENTS

The technical contents of the present invention will be described in detail with embodiments. However, it should be understood: these embodiments are only to exemplify the present invention but not to limit the scope of the present invention.

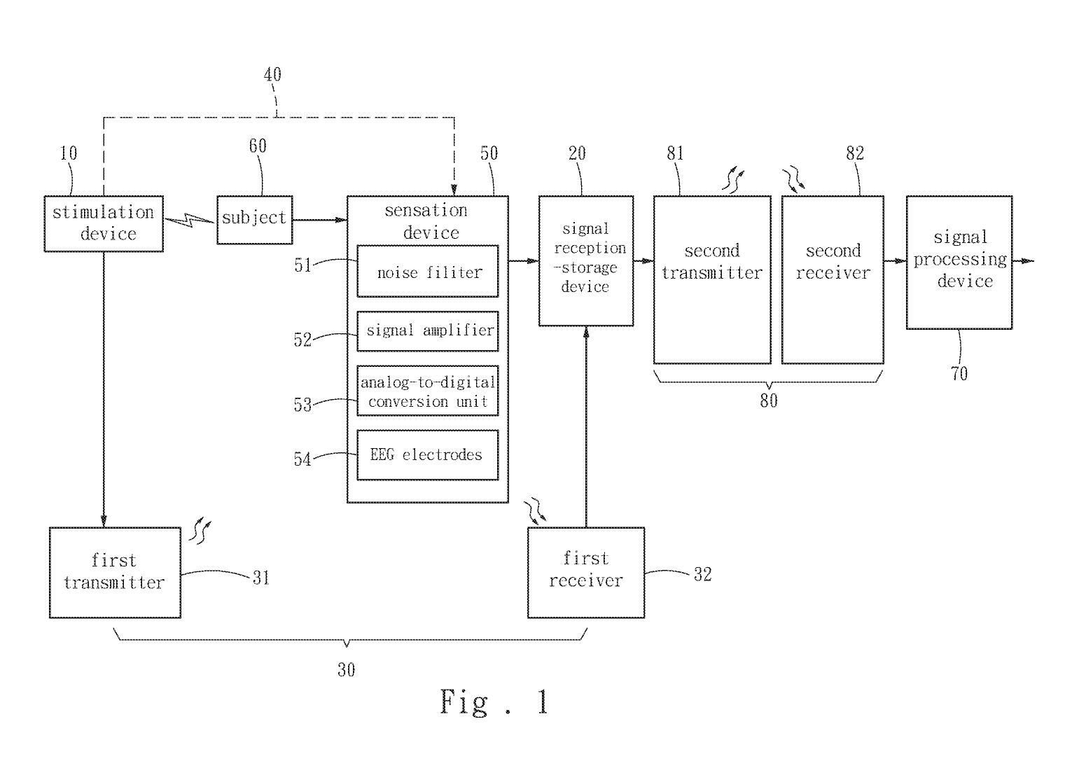

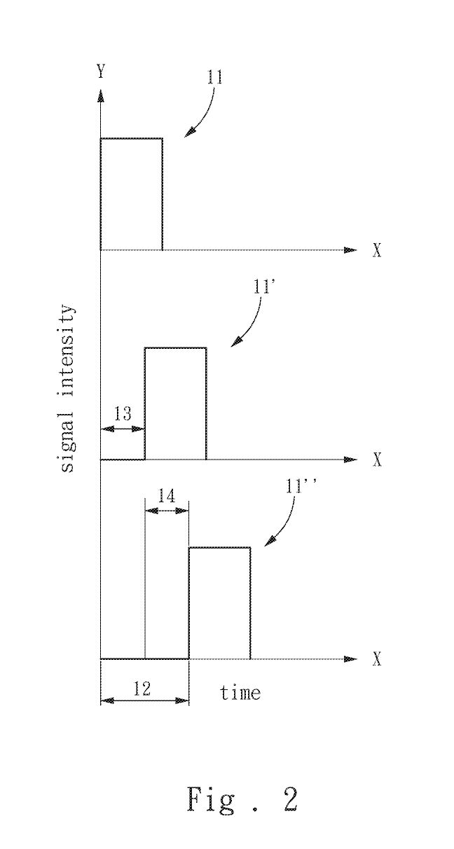

Refer to FIG. 1 and FIG. 2. FIG. 1 is a block diagram schematically showing a system realizing a method for measuring a biological stimulus signal according to one embodiment of the present invention. FIG. 2 is a diagram schematically showing a calibration signal according to one embodiment of the present invention. Firstly, a stimulation device 10 provides a calibration signal 11 and uses a wired transmission path and a wireless transmission path to transmit the calibration signal 11 to a signal reception-storage device 20. In one embodiment, the wireless transmission path is a first wireless transmission interface 30 linking the stimulation device 10 and the signal reception-storage device 20. In one embodiment, the wired transmission path is a wired transmission interface 40 connecting the stimulation device 10 with a sensation device 50, and the sensation device 50 is further connected with the signal reception-storage device 20. The signal reception-storage device 20 receives the calibration signals 11 respectively coining from the wireless transmission path and the wired transmission path and separately delayed by a wireless transmission time 12 and a wired transmission time 13 and then stores the calibration signal 11. The wireless transmission and the wired transmission respectively consume a wireless transmission time 12 and a wired transmission time 13. A time lag 14 exists between the wireless transmission time 12 and the wired transmission time 13.

Refer to FIG. 2, wherein the X axis denotes time and the Y axis denotes the signal intensity. In FIG. 2, the charts from top to bottom respectively show the calibration signal 11 sent out by the stimulation device 10, the calibration signal 11′ received by the signal reception-storage device 20 through the wired transmission path, and the calibration signal 11″ received by the signal reception-storage device 20 through the wireless transmission path. As shown in FIG. 2, the difference between the wireless transmission time 12 and the wired transmission time 13 is exactly the time lag 14. In other words, the time lag 14 is the difference between the time of transmitting a signal through the sensation device 50 and the time of transmitting a signal through the first wireless transmission interface 30.After the time lag 14 is acquired, the wired transmission interface 40 is removed.

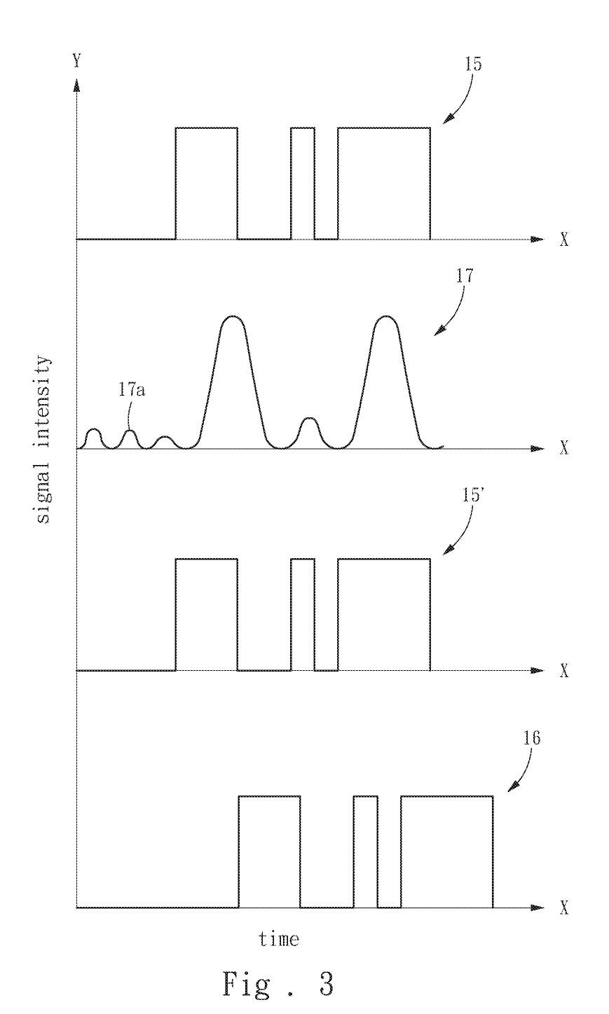

Refer to FIG. 1 and FIG. 3. Next, the stimulation device 10 provides a stimulus signal 15 to a subject 60. At the same time, the stimulation device 10 transmits a synchronization signal 15′ to the signal reception-storage device 20 through the first wireless transmission interface 30. The signal reception-storage device 20 stores the synchronization signal 15′ as a comparison signal 16. The synchronization signal 15′ may be completely the same as the stimulus signal 15 (as shown in the drawing). The synchronization signal 15′ may be a pulse signal, which is also used to denote the initial time point of the stimulus signal 15.

Next, the subject 60 receives the stimulus signal 15 and generates a response. The sensation device 50 detects and converts the response of the subject 60 and transmits the response to the signal reception-storage device 20. The signal reception-storage device 20 stores the response as a response signal 17, as shown in FIG. 3. The response signal 17 normally has a fundamental wave 17a.The fundamental wave 17a is a wave normally generated by the subject 60 and unrelated to the stimulus signal 15. The response signal 17 has the significant wave that is the response of the subject 60 to the stimulus signal 15.

Then, let a signal processing device 70, which is connected with the signal reception-storage device 20, work out the time lag 14 according to the wireless transmission time 12 and the wired transmission time 13, and use the time lag 14 to calibrate either of the comparison signal 16 and the response signal 17. After calibration, the signal processing device 70 integrates the comparison signal 16 and the response signal 17 to generate a synchronized integrated data of the subject 60.

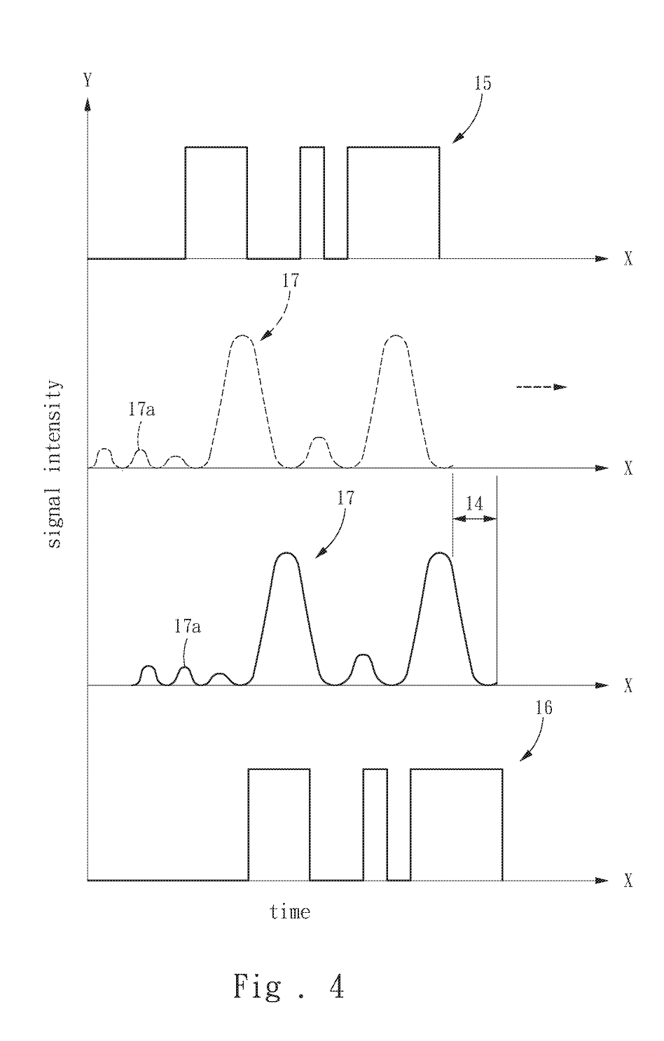

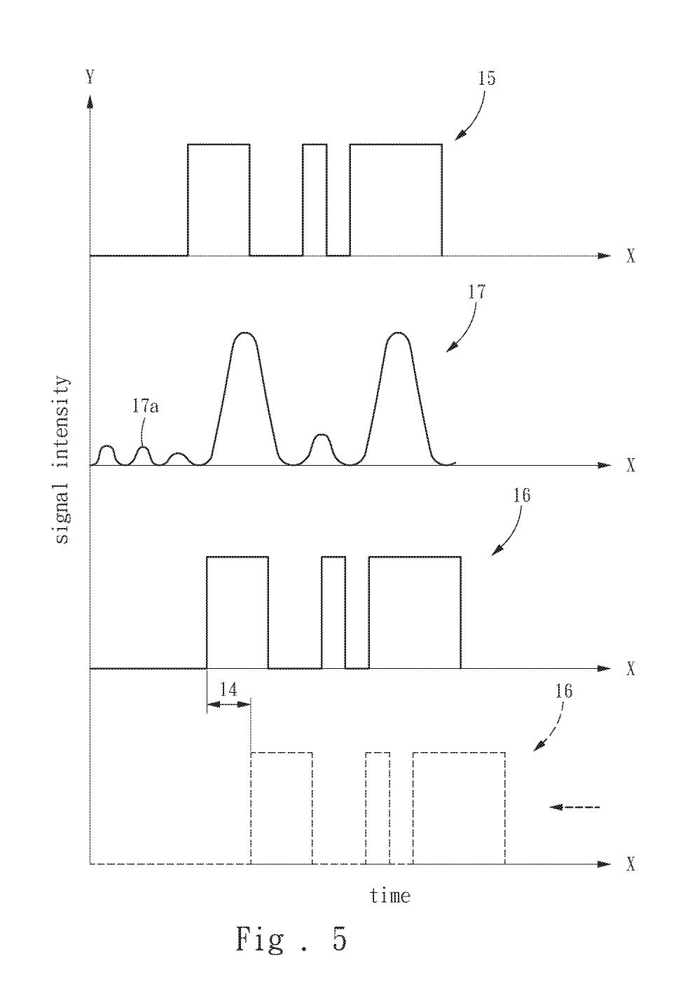

Refer to FIG. 4 and FIG. 5. The present invention calibrates the comparison signal 16 and the response signal 17 in a delay way (shown in FIG. 4) or a compensation way (shown in FIG. 5). As shown in FIG. 4, the signal processing device 70 delays the response signal 17 according to the time lag 14 to calibrate the response signal 17, wherein the response signal 17 is delayed (displaced rightward) in the time axis (the X axis) by the time lag 14, wherein the dotted curve denotes the uncalibrated response signal 17. Thereby, the calibrated response signal 17 is correctly corresponding to the comparison signal 16 in the time domain. After integration, the synchronized integrated data of the subject 60 is generated.

As shown in FIG. 5, the signal processing device 70 compensates the comparison signal 16 according to the time lag 14 to calibrate the comparison signal 16, wherein the comparison signal 16 is compensated (displaced leftward) by the time lag 14 in the time axis (the X axis), and wherein the dotted curve denotes the uncalibrated comparison signal 16. Thereby, the calibrated comparison signal 16 is correctly corresponding to the response signal 17. After integration, the synchronized integrated data of the subject 60 is generated.

In the present invention, the first wireless transmission interface 30 is based on a wireless technology selected from a group including the technologies of WiFi, WiMAX, LTE, UWB, ZigBee, Bluetooth, microwave, infrared, etc. The scope of the present invention also covers the first wireless transmission interface 30 based on other technologies not listed above but able to transmit data wirelessly. The first wireless transmission interface 30 includes a first transmitter 31 connected with the stimulation device 10 and a first receiver 32 connected with the signal reception-storage device 20.

In one embodiment, the signal reception-storage device 20 is connected with the signal processing device 70 through a second wireless transmission interface 80. Similarly, the second wireless transmission interface 80 is based on a wireless technology selected from a group including the technologies of WiFi, WiMAX, LTE, UWB, ZigBee, Bluetooth, microwave, infrared, etc. The second wireless transmission interface 80 includes a second transmitter 81 connected with the signal reception-storage device 20 and a second receiver 82 connected with the signal processing device 70.

In one embodiment, the sensation device 50 includes a noise filter 51, a signal amplifier 52, an analog-to-digital conversion unit 53, and an EEG(Electroencephalography) electrodes 54 contacting the head (not shown in the drawings) of the subject 60. In this embodiment, the response measured by the sensation device 50 is a brain wave from the head. In the sensation device 50, the EEG electrodes 54 detect the brain wave; the noise filter 51 filters out noise signals; the signal amplifier 52 amplifies the signal; the analog-to-digital conversion unit 53 converts the signal into a digital style to transmit.

Below is introduced one of the embodiments of the present invention. However, the present invention is not limited by this embodiment.

In this embodiment, the stimulation device 10 is an audio-video device able to generate specified images or sounds. The sensation device 50 is worn by the subject 60, using the EEG electrodes 54 thereof to acquire the brain wave signal.

Firstly, before the subject 60 wears the sensation device 50, the time lag 14 is acquired from the wireless transmission path and the wired transmission path in advance. In other words, the time lag 14 is the difference of the time that a signal passes through the sensation device 50 and the time that the signal passes through the first wireless transmission interface 30. The time lag 14 can be applied to the tests for different subjects 60 without re-measurement unless there is variation in the wireless transmission path and/or the wired transmission path.

After the time lag 14 is acquired, the wired transmission path is removed. Next, let the subject 60 wear the sensation device 50 and let the stimulation device 10 provide the subject 60 with the stimulus signal 15 in form of images or sounds. For example, the stimulation device 10 outputs the stimulus signal 15 to an image display or a speaker to drive the image display or the speaker to generate images or sounds. At the same time, the stimulation device 10 outputs a synchronization signal 15′ through the first wireless transmission interface 30to the signal reception-storage device 20 as the comparison signal 16. Then, the sensation device 50 detects the brain wave of the subject 60 and transmits it to the signal reception-storage device 20 to store as the response signal 17.

Next, use the time lag 14 to calibrate either of the comparison signal 16 and the response signal 17. After calibration, the comparison signal 16 and the response signal 17 are integrated to generate the synchronized integrated data of the subject 60. In the cases that the stimulation device 10 demands the subject 60 to undertake displacements or significant body movements, the design of the first wireless transmission interface 30 and the second wireless transmission interface 80 of the present invention can exempt the displacements or movements of the subject 60 from interference or limitation.

In comparison with the conventional technology, the present invention has the following advantages:
1. The present invention adopts a wireless transmission technology, exempting the subject from being constrained in the space and distance of movements, applying to more types of tests, and increasing the convenience of usage.
2. The present invention uses the time lag to undertake calibration, avoiding the problem caused by the difference between the time of transmitting a signal wirelessly and the time of passing the signal through the sensation device, and correctly generating the synchronized integrated data of the subject to satisfy demands in applications.
3. The present invention connects the second wireless transmission interface with the signal processing device arranged in a far end, exempting the subject from being constrained by the signal processing device, enabling the subject to move free, and increasing the comfort of the subject.
4. The present invention arranges the noise filter, signal amplifier and analog-to-digital conversion unit directly in the sensation device, undertaking wave filtration, amplification, and analog-to-digital conversion, and reducing interference from the external environment.

What is claimed is:

1. A method for measuring a biological stimulus signal, comprising steps:
letting a stimulation device provide a calibration signal and use a wireless transmission path and a wired transmission path to transmit the calibration signal to a signal reception-storage device, wherein the signal reception-storage device receives the calibration signals respectively coining from the wireless transmission path and the wired transmission path and separately delayed by a wireless transmission time and a wired transmission time, and wherein a time lag exists between the wireless transmission time and the wired transmission time;
letting the stimulation device provide a stimulus signal to a subject and simultaneously transmit a synchronization signal to the signal reception-storage device through a first wireless transmission interface, wherein the signal reception-storage device stores the synchronization signal as a comparison signal;
after the subject receives the stimulus signal and generates a response, letting a sensation device detects and converts the response and transmit the response to the signal reception-storage device to store as a response signal; and
letting a signal processing device, which is connected with the signal reception-storage device, use the time lag to calibrate either of the comparison signal and the response signal, and integrate the comparison signal and the response signal after calibration to generate a synchronized integrated data of the subject.

2. The method for measuring a biological stimulus signal according to claim 1, wherein the signal processing device delays the response signal according to the time lag to calibrate the response signal.

3. The method for measuring a biological stimulus signal according to claim 1, wherein the signal processing device compensates the comparison signal according to the time lag to calibrate the comparison signal.

4. The method for measuring a biological stimulus signal according to claim 1, wherein the first wireless transmission interface is based on a wireless technology selected from the group including the technologies of WiFi, WiMAX, LTE, UWB, ZigBee, Bluetooth, microwave, and infrared.

5. The method for measuring a biological stimulus signal according to claim 1, wherein the first wireless transmission interface includes a first transmitter connected with the stimulation device and a first receiver connected with the signal reception-storage device.

6. The method for measuring a biological stimulus signal according to claim 1, wherein the signal reception-storage device is connected with the signal processing device through a second wireless transmission interface.

7. The method for measuring a biological stimulus signal according to claim 6, wherein the second wireless transmission interface is based on a wireless technology selected from the group including the technologies of WiFi, WiMAX, LTE, UWB, ZigBee, Bluetooth, microwave, and infrared.

8. The method for measuring a biological stimulus signal according to claim 6, wherein the second wireless transmission interface includes a second transmitter connected with the signal reception-storage device and a second receiver connected with the signal processing device.

9. The method for measuring a biological stimulus signal according to claim 1, wherein the sensation device includes a noise filter, a signal amplifier, and an analog-to-digital conversion unit, and wherein after the sensation device acquires the response from the subject, the noise filter filters out noise signals of the response; the signal amplifier amplifies the signal from the noise filter; the analog-to-digital conversion unit converts the signal from the signal amplifier into a digital response signal.

10. The method for measuring a biological stimulus signal according to claim 9, wherein the sensation device further includes EEG electrodes contacting the head of the subject, and wherein the response of the subject is a brain wave from the head, and wherein the EEG electrodes detect the brain wave.

11. The method for measuring a biological stimulus signal according to claim 1, wherein the wireless transmission path includes the first wireless transmission interface linking the stimulation device and the signal reception-storage device, and wherein the wired transmission path includes a wired transmission interface connecting the stimulation device withthe sensation device, and the sensation device is further connected with the signal reception-storage device.
